(19) United States
(12) Patent Application Publication
(10) Pub. No.: US 2011/0034762 A1
(43) Pub. Date: Feb. 10, 2011

(54) GASTRIC RING INCLUDING A ONE-PIECE BELT

(75) Inventor: Pascal Paganon, Rhone (FR)

Correspondence Address: OCCHIUTI ROHLICEK & TSAO, LLP, 10 FAWCETT STREET, CAMBRIDGE, MA, 02138, US

(73) Assignee: COMPAGNIE EUROPEENNE D'ETUDE ET DE RECHERCHE DE DISPOSITIFS POUR L'IMPLANTATION PAR LAPAROSCOPIE, VIENNE (FR)

(21) Appl. No.: 12/937,112

(22) PCT Filed: Apr. 10, 2009
(86) PCT No.: PCT/FR09/50670

§ 371 Date: Oct. 8, 2010

(30) Foreign Application Priority Data
Apr. 14, 2008 (FR) 08 02033

(51) Int. Cl. A61F 2/04; B27N 3/08

(52) U.S. Cl. 600/37; 606/151; 264/239

(57) ABSTRACT

The invention relates to an implantable surgical ring (1) to go around a biological organ and constituting a pocket or duct designed for adjustment of the section through which said organ passes, said ring (1) including a belt (2) comprising at least one locking means (3) for said ring (1) in the closed functional position thereof around said biological organ and a flexible inflatable band (10) comprising at least one opening (11) enabling the supply therein of an inflation fluid, said belt (2) being fitted with the flexible inflatable band (10) and attached to the latter, said ring being characterized in that the belt (2) includes a coupling means (14) for coupling said at least one opening (11) of the flexible inflatable band (10) with an inflation fluid supplying means (15).

### TECHNICAL FIELD

The present invention pertains to the general technical field of surgical implants designed to be implanted in a patient's body around a biological organ forming a pocket or a duct.

The present invention relates especially to ring-type implants designed to form a closed loop around a biological organ constituting a pocket or a duct to reduce the diameter of said biological organ.

The present invention preferably pertains to a gastroplasty ring but is also to aimed at providing a ring designed for use in the treatment of urinary incontinence or fecal incontinence (entailing an artificial sphincter) or again as a ring designed to regulate the blood flow in blood vessels, this list being in no way restrictive.

The invention pertains more particularly to a surgical ring implantable in a is human or animal body, said ring being designed to go around a biological organ and constituting a pocket or a duct in order to modify the section of passage of said organ, said ring comprising firstly a belt with at least one means for locking said ring in a closed functional position around said biological organ and secondly a flexible, inflatable band comprising at least one ring-shaped gripping chamber designed to receive an inflation fluid and comprising at least one aperture enabling the supply therein of inflating fluid, said belt being brought to the flexible inflatable band and being fixed to this band.

The present invention also pertains to a method for manufacturing a surgical ring implantable in a human or animal body, said ring being designed to go around a biological organ and constituting a pocket or a duct in order to modify the section of passage of said organ, said method comprising a step for manufacturing a belt with at least one means for locking said ring in a closed functional position around said biological organ and secondly a flexible inflatable band comprising at least one ring-shaped gripping chamber designed to receive an inflation fluid and comprising at least one aperture enabling the supply therein of inflating fluid, as well as a step for manufacturing the belt with the flexible inflatable band, said belt being brought to the flexible inflatable band and being fixed to this band.

### PRIOR ART

There are known ways of making use of implantable devices in order to reduce the volume of the stomach or the diameter of its stoma in such a way as to facilitate the loss of weight of a patient in the context of a treatment for morbid obesity. Indeed, when conventional methods of is dieting prove to be ineffective and/or when the patient's obesity proves to be particularly dangerous for his or her health (entailing cardiovascular problems, joint ailments, diabetes etc), it is preferable to use surgical methods for implanting devices designed to reduce food intake.

These known methods include the possibility of going in for gastric constriction by means of a gastroplasty ring designed to be implanted around the stomach in a surgical operation so as to appreciably reduce the diameter of the passage for food into the stomach.

There is also a known gastroplasty ring comprising firstly a belt-forming dorsal band and secondly a flexible inflatable band attached and fixed on to the belt, along this belt, to form a unitary gastric ring. The ring is shut by means of an appropriate locking device positioned at the ends of the belt and enabling the ring to grip the stomach functionally according to an appreciably circular configuration.

The main function of the dorsal band is to contain the deformation of the flexible inflatable band so as to promote a predominantly centripetal inflation of this band, going towards the stomach.

The volume of the inflatable band is adjusted by adding or removing inflation fluid. To this end, the prior-art ring referred to here above is provided with a catheter connected at one of its ends to the flexible inflatable band of the ring and at the other end to a subcutaneous implantable site used to introduce or remove inflatable fluid through the patient's skin by means of a hollow needle piercing the impervious membrane of the implantable site.

Such a ring has a certain number of drawbacks.

Thus, in this prior-art ring the catheter projects out of the flexible inflatable band, near the stomach, and this is liable to hamper the surgeon when the ring is being handled in order to be placed around the stomach. Indeed, the surgeon may have to take special precautions in order to release the catheter fully from the ring, especially in order to avoid getting it stuck between the flexible inflatable band and the stomach.

Furthermore, the entry of the catheter to the flexible inflatable band in proximity to the stomach favors the rubbing of the catheter against the stomach or even its retention in folds, if any, in the stomach, thus prompting necrosis in the outer lining of the stomach and making the ring particularly uncomfortable for the patient.

Furthermore, this type of ring entails a joining of the catheter to the flexible inflatable band by a simple fastening point which can be greatly stressed by friction between the catheter and the stomach and the surrounding tissues. Such friction can embrittle the attachment of the catheter and even cause its possible deterioration and lead the catheter to get disconnected in untimely fashion

Such a fastening of the catheter to the flexible inflatable band may hamper the homogenous inflation of the ring and could for example cause possible folds to be formed in the flexible inflatable band. This type of catheter fastening therefore does not prove to be a reliable means of inflating the ring in a simple and sure way.

In addition to the above-mentioned operating difficulties, there may be to possible problems of manufacture. Indeed, the manufacturing of such a ring proves to be particularly complicated, especially when assembling the catheter with the inflatable band. In general, the inflatable band is made out of an elastomer material that is less rigid than the catheter, and this results in a particularly delicate and lengthy assembling operation, is during which human intervention may be necessary to achieve stable and impervious connection of the catheter to the inflatable band. This step appreciably burdens the manufacturing method and contributes to making it costly.

Naturally, such drawbacks may call for further surgical operations, for example to remove a deteriorated ring, and this unnecessarily mobilizes medical staff, makes the processing operation more burdensome, causes additional difficulties for the patient and even exposes him to post-operation complications.

### SUMMARY OF THE INVENTION

The objects assigned to the present invention are therefore aimed at remedying the above-mentioned drawbacks and proposing a new implantable surgical ring that is easy to implement for the surgeon, has an non-traumatic character, is robust and easy in its construction and costs little to manufacture on an industrial scale.

Another object assigned to the present invention is aimed at proposing a novel implantable surgical ring with particularly simple and easily implemented operation.

Another object assigned to the present invention seeks to propose a novel implantable surgical ring which is fast and simple to implement.

Another object of the present invention is aimed at proposing a novel implantable surgical ring ensuring the security and imperviousness of the inflation.

Another object assigned to the present invention is aimed at proposing a novel implantable surgical ring held in a stable and lasting way when in functional position.

Another object assigned to the present invention proposes a novel implantable surgical ring that has a non-traumatic configuration and is particularly adapted to the surrounding tissues.

Another object assigned to the present invention is aimed at proposing a novel implantable surgical ring of simple design for which the joining of the constituent parts is facilitated.

Another object assigned to the present invention seeks to propose a novel implantable surgical ring enabling simple and functional inflation without any leakage of inflation fluid.

Another object assigned to the present invention seeks to propose a novel implantable surgical ring with homogenous inflation that limits the folds.

Another object assigned to the present invention seeks to propose a novel implantable surgical ring that has a simple, light, easily implemented and low-cost structure.

Another object assigned to the present invention seeks to propose a novel implantable surgical ring used for the treatment of obesity.

Another object assigned to the present invention seeks to propose a novel method for making an, implantable surgical ring that is easy and low-cost to achieve on an industrial scale, and makes said ring easy to implement by the surgeon, non-traumatic in character and robust in construction.

Another object assigned to the invention seeks to propose a novel method of manufacturing an implantable surgical ring which facilitates the joining and the stable holding of said ring in the functional position.

Another object assigned to the invention seeks to propose a novel is method for making an implantable surgical ring that is simple, costs little and is fast to implement.

Another object assigned to the invention seeks to propose a new method for manufacturing an implantable surgical ring giving the ring excellent stability in a functional position.

Finally, a last object assigned to the invention seeks to propose a new method for manufacturing an implantable surgical ring for gastric use.

The objects assigned to the invention are achieved by means of an surgical ring implantable in a human or animal body, said ring being designed to go around a biological organ constituting a pocket or a duct in order to modify the section of passage of said organ, said ring comprising firstly a belt comprising at least one means for locking said ring in a closed functional position about said biological ring and secondly a flexible inflatable band comprising at least one ring-shaped gripping chamber designed to receive an inflation fluid and comprising at least one aperture enabling the supply therein of inflation fluid, said belt being brought to the flexible inflatable band and fixed to it, said ring being characterized in that the belt comprises a means for placing said at least one aperture of the flexible inflatable band in communication with an inflation fluid supplying means.

The objects assigned to the invention are also achieved by means of a method for manufacturing a surgical ring implantable in a human or to animal body, said ring being designed to go around a biological organ constituting a pocket or a duct in order to modify the section of passage of said organ, said method comprising a step for manufacturing a belt comprising at least one means for locking said ring in a closed functional position about said biological ring, a step for manufacturing a flexible is inflatable band comprising at least one ring-shaped gripping chamber designed to receive an inflation fluid and comprising at least one aperture enabling the supply therein of inflation fluid, as well as a step for assembling the belt with the flexible inflatable band, said belt being brought to the flexible inflatable band and fixed to it, said method for manufacturing being characterized in that the step for manufacturing the belt comprises a sub-step for manufacturing a means for placing said at least one aperture of the flexible inflatable band in communication with an inflation fluid supply means.

### BRIEF DESCRIPTION OF THE DRAWINGS

Other objects, characteristics and advantages of the invention shall appear in greater detail from the following description as well as from the appended drawings, given purely on an illustratory and non-restrictive basis, of which:

FIG. 1 is an illustration, in a perspective view in profile, of a surgical ring according to the invention in its open position.

FIG. 2 is an illustration in a side view of a surgical ring according to the invention in its closed position.

FIG. 3 is an illustration, in an exploded perspective view in profile, of the surgical ring of FIG. 1 according to a first embodiment.

FIG. 4 is an illustration, in an exploded perspective view in profile, to of the surgical ring of FIG. 1 according to a second embodiment.

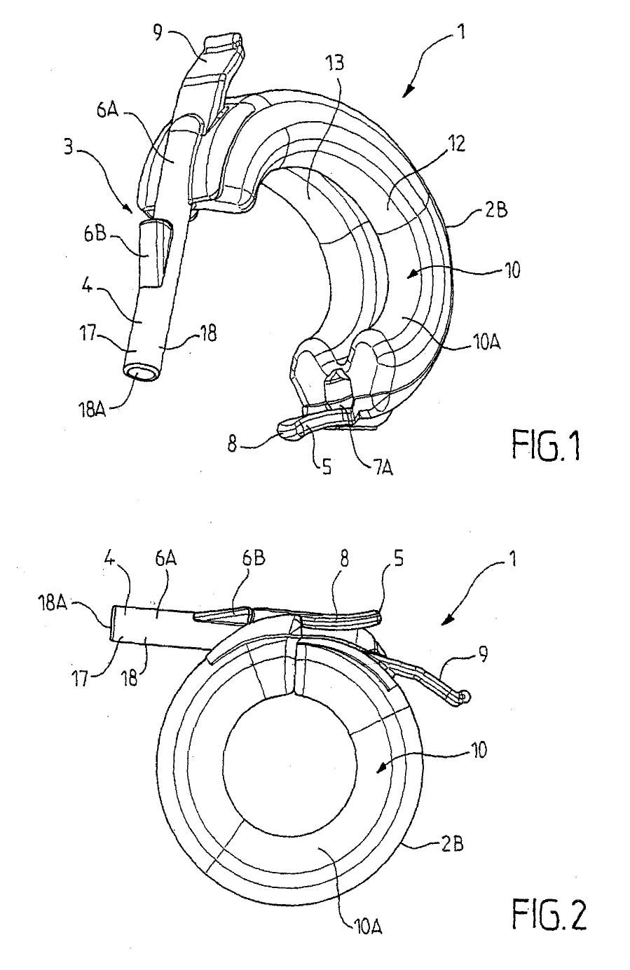

### BEST MANNER OF OBTAINING THE INVENTION

In the following description, by way of an example and for reasons of convenience, we shall attempt to describe a gastric ring (or gastroplasty ring) designed to surround the stomach in order to reduce the section of is passage of this stomach in the context of a treatment for obesity.

The present invention however is not limited to a gastric application of the ring and also generally covers surgical rings designed to be implanted in a patient's body around a biological organ and constituting a pocket or a duct in order to modify the section of passage of said organ when this organ is gripped by the ring.

Thus, the present invention can for example be used in the context of a treatment of urinary or fecal incontinence or again for the regulation of the blood flow.

Depending on the use made of it, the present ring could have dimensions, a shape and functions adapted to the organ concerned by the constriction, such as for example the bladder, the urethra, the intestine, an artery, a vein etc.

The present invention therefore pertains to a surgical ring 1 implantable in a human or animal body, preferably a gastric ring 1. Said ring 1 is designed to go around a biological organ constituting a pocket or a duct, especially the patient's stomach, in order to modify the section of passage of said organ, in particular to reduce the stoma of the stomach and thus limit the quantity of food ingested by the patient.

The ring 1 is made out of several constituent parts joined together to form to a unitary functional ring 1. The ring 1 comprises firstly a belt 2, designed to form the outermost part of the ring 1, namely the part at the greatest distance from the stomach in contact with the surrounding tissues. The belt 2 is made of an elastomer material, preferably silicone, advantageously having a circular shape memory. Thus, the belt 2 takes is the form that is substantially rounded in the open position (at rest), thus fostering its shaping around the stomach.

The belt 2 has an internal face 2A defining the inner perimeter of the belt 2, i.e. the part closest to the stomach, as well as an external face 2B defining the outer perimeter of the belt, i.e. the one that is at the greatest distance from the stomach and will come into contact with the surrounding tissues.

The belt 2 has at least one means 3 for locking said ring in a closed functional position around said biological organ. The locking means 3 comprise at least two closing elements 6, 7 designed to cooperate with each other to ensure the locking of the ring 1 in its closed configuration around the stomach, the closing elements 6 and 7 being preferably positioned at the ends 4 and 5 respectively of the belt 2.

The closing element 7 advantageously has a sleeve shape 7A designed to receive the closing element 6 forming a rod 6A. These two closing elements 6, 7 are designed to be fitted into each other in a stable manner, especially through the presence of at least one snug 6B present on the rod 6A. Indeed, the snug 6B preferably has an insertion face facilitating the passage of the rod 6A, by elastic deformation, into the sleeve 7A and a non-return face which prevents the untimely opening of the ring 1 once the rod 6A has been introduced into the sleeve 7A beyond the snug 6B.

In the closed position, when the closing elements 6, 7 of the locking to means 3 cooperate with one another, the belt 2 has an appreciably rounded shape that preferably is appreciably circular.

Furthermore, the belt 2 of the ring can be provided with one or more locking/unlocking tongues 8 designed to facilitate the handling of the ring 1 by laparoscopy, especially when it is being closed and/or being opened. Indeed, the locking/unlocking tongue 8 is preferably formed by the end 5 of the belt 2 and can be used by the surgeon by means of a grasping instrument to close and/or open the ring 1 by pulling on it.

Advantageously, the belt 2 furthermore has one or more grasping tongues 9 also designed to facilitate the use of the ring 1 in the patient's body. Indeed, through a grasping tongue 9 of this kind, the surgeon can easily handle the ring, especially when closing and/or opening it.

The ring 1 furthermore has a flexible inflatable band 10 comprising at least one aperture 11 enabling the supply therein of an inflation fluid. Preferably, the flexible inflatable band 10 is independent of the belt 3 and forms a pocket, i.e. a closed and individualized chamber. The inflatable band 10 therefore advantageously corresponds to a flexible gut forming a pocket that is impervious to the inflation fluid.

Advantageously, the inflation fluid is a gas or a liquid, preferably a physiological serum. The flexible inflatable band 10 is preferably made out of elastomer, advantageously silicone, with an appreciably circular shape memory.

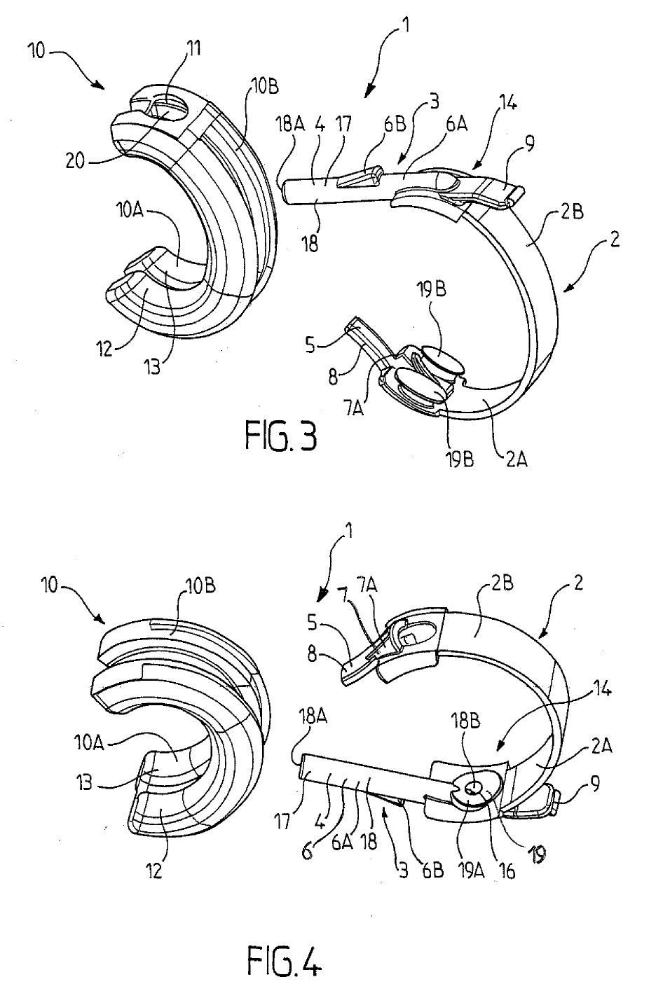

The flexible inflatable band 10 has an internal face 10A in contact with the stomach and an opposite external face 10B. The flexible inflatable band 10 also has at least one ring-shaped gripping chamber 12 designed to receive the inflation fluid. The chamber 12 therefore advantageously enables a substantially ring-shaped gripping to be achieved around the organ, preferably the stomach. Preferably, the flexible inflatable band 10 has at least two ring-shaped gripping chambers 12, 13 which extend substantially in parallel to each other, as can be seen in FIGS. 3 and 4. Said chambers 12, 13 are advantageously staged relative to each other, and receive inflation fluid by the aperture 11 prepared advantageously on is the external face 10B of the flexible inflatable band 10.

The supply of inflation fluid into the flexible inflatable band 10 modifies the volume of the ring-shaped gripping chambers 12, 13 so as to enable the efficient gripping of the stomach.

Without departing from the framework of this invention, it can also be envisaged that the flexible inflatable band 10 will include, on its external face 10B, several apertures 11 enabling the inlet of inflation fluid.

The belt 2 is attached to the flexible inflatable band 8 outside this band and is fixed to the flexible inflatable band 8. Indeed, as illustrated in the figures, the internal face 2A of the belt 2 is positioned on the external face 10B of the flexible inflatable band 10. In other words, the flexible inflatable band 10 and the belt 2, which are perfectly distinct, separated, individualized and independent of each other, are assembled on and throughout the length of the external face 10B and internal face 2A respectively. Preferably, the fastening of the flexible inflatable band 10 against the belt 2 and along it is uniform in such a way that the flexible inflatable band 10 and the belt 2 are substantially coextensive.

Advantageously, the internal face 2A of the belt 2 has a shape that is substantially complementary to that of the external face 10B of the flexible inflatable band 10 against which said belt 2 is assembled. Preferably, the internal face 2A of the belt 2 comprises a raised band (not shown) throughout its length, advantageously having the shape of a rectangular parallelepiped designed to fit into a hollow band that is present throughout the length of the external face 10B of the flexible inflatable band 10 and has a shape substantially complementary to that of the raised band of the belt 2, i.e. having the shape of a hollow rectangular parallelpiped. Such a complementarity of shape between the faces 2A and 10B facilitates the stable assembling of the belt 2 with the flexible inflatable band 10.

The belt 2 fixed to the band 10 facilitates a centripetal inflation of the latter once the ring has been implanted around the stomach so as to efficiently clamp the stomach. Indeed, the belt 2 plays a dorsal role to the flexible inflatable band 10. Furthermore, the assembling of said belt 2 with the exterior of the flexible inflatable band 10 to make the ring 1 has the advantage of enabling the use of materials of different natures for each of the elements, namely said belt 2 and band 10. In one particular advantageous embodiment, the belt 2 is made out of an elastomer material having higher rigidity and lower elasticity than the elastomer constituting the band 10 so as to facilitate the centripetal inflation of the ring when inflation fluid is added into the band 10.

The belt 2 of the present invention comprises a means for placing at least one aperture 11 of the flexible inflatable band 10 in communication with an inflation fluid supplying means 15. Advantageously, the inflation fluid is brought to the level of the belt 2 which then provides for the transit up to the flexible inflatable band 10 through a communications set-up means 14. The belt 2 therefore acts as an interface for the transmission of inflation fluid from the implantable site to the flexible inflatable band 10. In other words, the inflating fluid advantageously travels through the belt 2 before reaching the flexible inflatable band 10.

In a preferred embodiment of the present invention, the inflation fluid supplying means 15 (not shown in the figures) comprise a conduit, preferably a catheter. In general, the catheter is connected to a subcutaneous implantable site enabling the supply and/or withdrawal of inflation fluid by means of a needle inserted into the site and connected to a syringe. In other words, the catheter is connected to the belt 2 and fluid is transferred between the catheter and the belt 2 at the communications set-up means 14. The inflation fluid therefore travels through the belt 2 is before reaching the flexible inflatable band 10.

Advantageously, these communications set-up means 14 comprise a hole 16, made in the belt, that provides for communication of fluids with said aperture 11 of the flexible inflatable band 10. Preferably, the hole 16 of the belt 2 is brought to the aperture 11 of the flexible inflatable band 10 so as to set up impervious communication of fluids between said band 10 and said belt 2. In other words, the hole 16 made in the belt 2, preferably at its internal face 2A, is joined to the aperture 11 so as to set up communication of fluids between the belt 2 and the band 10.

Advantageously, the hole 16 of the belt 2 also enables communication of fluids between the flexible inflatable band 10 comprising at least two ring-shaped gripping chambers 12, 13 and the inflation fluid supply means 15. This fluid communication is set up by using the communications set-up means 14 for the belt 2 which firstly receive the catheter bringing the inflation fluid and secondly transfer the inflation fluid to the flexible inflatable band 10 through the hole 16 brought to the aperture 11 of said band 10. The inflation fluid is thus advantageously transferred through the belt 2 into the inflatable band 10 and therefore goes through said belt 2.

Preferably, the hole 16 of the belt 2 also provides for communication of fluids between each of the gripping chambers 12, 13. Indeed, the impervious contact between the hole 16 of the belt 2 and the aperture 11 of the flexible inflatable band 10 advantageously provides for the creation of a fluid transfer zone 20 enabling the filling of each of the ring-shaped gripping chambers 12, 13 as well as communication of fluids between these two chambers 12, 13. This zone 20 has the useful feature by which it ensures firstly a homogenous inflation of the ring 1 after it has been implanted around the stomach and secondly its smooth deflation at the end of use. Furthermore, the zone 20 facilitates the balancing of the volume of these two chambers 12, 13 during use in enabling the transfer of fluid from one chamber to the other.

Alternately, in another embodiment, it can also be envisaged that the two chambers 12, 13 will be completely separated in fluidic terms, i.e. that no transfer of fluid is possible between said chambers 12, 13, for example because of an intermediate partition wall between the chambers 12, 13 throughout their length. In this case, each chamber 12, 13 has a hole 16 by which inflation fluid is supplied and/or removed. The transfer of inflation fluid can be done using a single catheter linked to both chambers 12, 13 or by means of two catheters each linked to one chamber and enabling independent management of each of said chambers 12, 13.

Preferably, the communications set-up means 14 comprise a conduit 17 which is connected firstly to the hole 16 and secondly to the inflation fluid supply means 15, i.e. to the catheter. Advantageously, the conduit 17 is substantially indistinguishable from the rod 7 forming a closing element for the locking means 3. The inflation fluid is indeed carried by the catheter to the conduit 17 and then transmitted by said conduit 17 to the flexible inflatable band 10 through the hole 16 prepared at the end of the conduit 17 on the belt 2. This conduit 17 which projects from the ring 1 when it is closed around the stomach, as illustrated in FIG. 3, thus facilitates the handling of the ring 1 by the surgeon and especially prevents the catheter from remaining against the stomach and/or hampering the surgeon when the ring 1 is being handled.

In a preferred embodiment, the communications set-up means 14 form a housing 18 which surrounds at least one part of said inflation fluid supply means 15, namely the catheter. As can be seen in the figures, the housing 18 is designed to receive the catheter (not shown), at its first end 18A, the catheter being inserted and stably fixed in the housing 18, and is also designed to open out at the level of its second 18B on the hole 16 assembled on the aperture 11 of the band 10. The housing 18 therefore enables the imperviously sealed transfer of inflation fluid from the catheter to the band 10.

Advantageously, the housing 18, by its pouch shape and its characteristics of flexibility related especially to the material that constitutes it, perfectly matches the shape of the catheter which is inserted into it, preferably by force. In order to ensure solid and lasting fastening of the catheter into the housing 18, the catheter is generally glued, preferably by coating the external surface of the part of the catheter inserted into the housing with a preferably biocompatible glue or adhesive material. Alternatively, it can also be envisaged to mold the catheter in the housing 18 of the belt 2 from a casting. The communications set-up means 14, especially the housing 18, therefore ensure a reliable fastening of the catheter to the belt 2. Furthermore, the housing 18 made out of elastomer material of the silicone type has a certain rigidity relative to the catheter making it possible to release the catheter from the stomach and thus prevent friction of the catheter with the stomach.

These communications set-up means 14 also facilitate the assembling of the belt 2 with the band 10. Indeed, the communications set-up means 14 comprise a fastening means 19 designed to cooperate with the aperture 11 of the flexible inflatable band 10 in order to contribute, at least locally, to the attaching of the belt 2 to the flexible inflatable band 10.

In a preferred embodiment, the fastening means 19 take the form of a button 19A whose configuration is appreciably complementary to that of the aperture 11 of the flexible inflatable band 10, said aperture 11 forming a buttonhole designed to receive said fastening means 19. As illustrated in FIG. 4, the button 19A of the belt 2 is advantageously pierced with the is hole 16 and inserted into the aperture 11 of the band 10, such a configuration providing firstly the assembling, at least locally, of the belt 2 with the band 10 and secondly, the communication of fluids between said belt 2 and said band 10.

The joining of the belt 2 to the band 10 at the fastening means 19, as in a buttoning or clip-on operation, advantageously enables the belt 2 to be held relative to the band 10 so as to facilitate their subsequent attachment by glueing.

It can also be envisaged, without in any way departing from the framework of the present invention, that the fastening means 19 will comprise several buttons 19A, 19B preferably positioned at each of the ends 4, 5 respectively of the belt 2. The button 19A comprising the hole 16 is inserted into the aperture 11 of the band 10 while the button or buttons 19B which are not pieced with a hole are inserted into two apertures (not shown) made on the internal face 10A of each of the ring-shaped gripping chambers 12, 13 of the band 10. Said at least three buttons 19A, 19B as illustrated in FIG. 3 facilitate the stable holding of the belt 2 on the flexible inflatable band 10 with a view to their subsequent fastening, for example by glueing, while at the same time, through the button 19A comprising the hole 16, setting up a simple and impervious communication of fluid between the belt 2 and the band 10.

In view of the above, the belt 2 of the ring 1 of the present invention therefore advantageously has the locking means 3, the grasping lug 9 and the communications set-up means 14 themselves comprising the hole 16, the conduit 17, the housing 18 and the fastening means 19.

Preferably, the belt 2 is formed by a part made as a single piece comprising firstly on its internal face 2A the hole 16 and secondly in the extension of one of its ends 4 the conduit 17 so as to form a single-piece is belt 2. Advantageously, the belt 2 is a single part comprising especially the communications set-up means 14, thus giving it a simple and functional single-piece configuration, which is particularly non-traumatic for the patient.

Indeed, the manufacturing of all the constituent elements of said belt 2 as as a single piece prevents the junction zones from possibly becoming abrasive to the stomach and the tissues. Furthermore, a single-piece structure of this kind prevents the untimely and potentially dangerous detachment of certain elements of the belt 2 which could freely move inside the human body.

In one advantageous embodiment of the invention, the flexible inflatable band 10 is also formed by a part that is made as one piece so as to form a single-piece flexible inflatable band 10 that is independent and separated from said belt 2. Exactly like the single-piece belt 2, the flexible inflatable band 10 forming a closed individual gut is formed by a single part comprising the above-mentioned constituent elements such as for example the aperture 11 and/or the hollow band on the external face 10B and/or the ring-shaped gripping chambers 12, 13.

According to the present invention, the configuration of the ring 1 in which the single-piece belt 2 is brought to and placed along the flexible inflatable band 10 outside the band 10 and fixed to this band 10 is particularly non-traumatic and easy to implement.

From a functional point of view, the ring 1 which comprises the flexible inflatable band 10, the belt 2 to which the catheter is attached at the housing 18, is introduced into the patient's body and is positioned so as to surround the patient's stomach. The surgeon closes the ring 1 around the stomach by introducing the catheter and the housing 18 into the sleeve 7A of the belt 2 until it goes beyond the snug 6A enabling the locking of is the appreciably circular ring 1 in its functional position. The catheter is then connected to a subcutaneous implantable site through which the surgeon supplies inflation fluid by means of a needle connected to syringe. This fluid travels through the catheter, reaches the housing 18 of the belt 2, crosses the hole 16 of the belt 2 and reaches the flexible inflatable band 10 at its aperture 11 communicating with the hole 16, the inflation fluid therefore travelling advantageously through the belt 2 and then reaching the inflatable band 10. The flexible inflatable band 10 then gets inflated centripetally so as to grip the stomach and reduce its stoma.

The present invention also relates to a method for manufacturing an implantable surgical ring in a human or animal body, said ring being intended to go around a biological organ constituting a pocket or duct in order to modify the section of passage of said organ. Preferably, the present invention relates to a method for manufacturing a gastric ring 1 as described here above, designed to surround the stomach in order to reduce its section of passage in the context of a treatment for obesity. The rest of this description will therefore strive to describe a method for manufacturing a gastric ring as defined here above.

The method comprises a step for manufacturing a ring 2 comprising at least one means 3 for locking said ring 1 in a closed functional position around said biological organ.

This method furthermore comprises a step for manufacturing a flexible inflatable band 10 comprising at least one ring-shaped gripping chamber 12 designed to receive an inflation fluid and comprising at least one aperture 11 enabling the supply therein of the inflation fluid, said flexible inflatable band 10 forming an independent closed gut that is distinct and separate from said belt 2.

After the belt 2 and the band 10 have been manufactured, the method of is the invention comprises a step for assembling the belt 2 with the flexible inflatable band 10, said belt 2 being connected against the flexible inflatable band 10 and fastened to this band 10. Advantageously, the operation for fastening the flexible inflatable band 10 against and along the belt 2 is done uniformly throughout the length of said band 10 and belt 2, so that these elements are substantially coextensive.

In a preferred embodiment, the step for manufacturing the belt 2 comprises a sub-step for manufacturing the internal face 2A of said belt 2, said internal face 2A having a shape that is appreciably complementary to that of the external face 10B of the flexible inflatable band 10 against which said internal face 2A of the belt 2 is attached, said external face 10B being manufactured during the step for manufacturing said inflatable band 10. Advantageously, a raised band is made on the internal face 2A of the belt 2, in the shape of a rectangular parallelpiped and with a shape appreciably identical to that of a hollow band made on the external face 10B of the band 10 so as to enable the inner face 2A of the belt 2 to be fitted to the external face 10B of the band 10 in a stable way by complementarity of shape.

Preferably, this stable assembling of the belt 2 with the flexible inflatable band 10 also implements a glueing operation during which the external face 10B of the inflatable band 10, preferably the external edges surrounding the above-mentioned hollow band, and/or the internal face 2A of the belt 2, preferably the external edges surrounding the above-mentioned raised band, receive a quantity of glue sufficient to ensure an efficient glueing of the band 10 with the belt 2.

The step for manufacturing the belt 2 of said manufacturing method also includes a sub-step for manufacturing a means 14 for placing said at least one aperture 11 of the flexible inflatable band 10 in communication with is an inflation fluid supply means 15, preferably a catheter as described here above.

Advantageously, the sub-step for manufacturing the communications set-up means 14 comprises an operation for manufacturing a fastening device 19 with a shape that is substantially complementary to that of the aperture 10 of the flexible inflatable band 10, said fastening device 19 being designed to provide for stable joining of said belt 2 with said flexible inflatable band 10 to form the ring 1.

Preferably, the step for manufacturing the flexible inflatable band 10 includes a sub-step for manufacturing at least two ring-shaped gripping chambers 12, 13 which extend appreciably in parallel to each other, the setting up of fluid communications between the flexible inflatable band 10 and the inflation fluid supply means 14 being done by the hole 16 of the belt 2.

Furthermore, the step for manufacturing the belt 2 is carried out advantageously by molding a part in one piece so as to form a single-piece belt 2 comprising firstly on its inner face 2A a hole 16 enabling communication of fluids with said aperture 11 of the flexible inflatable band 10 and comprising secondly, in its extension, the communications set-up means 14 forming a conduit 17, said conduit 17 being connected firstly to the hole 16 and secondly to the inflation fluid supply means 15, i.e. the catheter. Preferably, the molding of the belt 2 can also include the molding of the catheter in the conduit 17 from a casting so as to enable a reliable and imperviously sealed assembling of the catheter with the belt 2.

The molding step preferably brings into play a two-part mold in which a precise imprint is made of said belt 2 with its different constituent elements (the conduit 17, the housing 18, the fastening means 19, the is hole 16, the locking means 3 etc). The mold is then filled with the constituent material of the belt 2 in liquid form, preferably silicone, and closed. After cooling and demolding, this method is used to obtain a single-block belt comprising all the above-mentioned elements.

This single-piece molding of the belt 2 is particularly advantageous in terms of speed, cost and simplicity of manufacture. It restricts the number of steps needed to manufacture said belt 2 and provides for a non-traumatic configuration of this belt.

Furthermore, it can also be envisaged that the step for manufacturing the flexible inflatable band 10 will advantageously be done by molding a part as a single piece so as to form a single-piece flexible inflatable band 10. It is clearly quite possible, without departing from the framework of the present invention, to envisage the manufacture of the single-piece belt 2 and/or the single-piece flexible inflatable band 10 by a method other than that of molding.

The single-piece manufacture of the belt 2 and preferably of the flexible inflatable band 10 appreciably limits the number of steps of the method for manufacturing the ring 1, thus considerably reducing the time and costs of manufacturing the ring 1.

### POSSIBILITY OF INDUSTRIAL APPLICATION

The invention can be applied on an industrial scale to the designing and manufacture of implantable medical devices, especially for the treatment of obesity.

1. Surgical ring (1) implantable in a human or animal body, said ring (1) being designed to go around a biological organ constituting a pocket or a duct in order to modify the section of passage of said organ, said ring (1) comprising firstly a belt (2) comprising at least one means (3) for locking said ring (1) in a closed functional position about said biological ring and secondly a flexible inflatable band (10) comprising at least one ring-shaped gripping chamber (12) designed to receive an inflation fluid and comprising at least one aperture (11) enabling the supply therein of inflation fluid, said belt (11) being brought to the flexible inflatable band and fixed to it, said ring (1) being characterized in that the belt (2) comprises a means (14) for placing said at least one aperture (11) of the flexible inflatable band (10) in communication with an inflation fluid supplying means (15).

2. Surgical ring (1) according to claim 1, characterized in that these communications set-up means (14) comprise a hole (16) providing for communication of fluids with said aperture (11) of the flexible inflatable band (10).

3. Surgical ring (1) according to claim 1 or 2, characterized in that the communications set-up means (14) comprise a conduit (17) which is connected firstly to the hole (16) and secondly to the inflation fluid supply means (15).

4. Surgical ring (1) according to one of the claims 1 to 3, characterized in that the communications set-up means (14) form a housing (18) which surrounds at least one part of said inflation fluid supply means (15).

5. Surgical ring (1) according to one of the above claims, characterized in that the inflation fluid supplying means (15) comprise a conduit, preferably a catheter.

6. Surgical ring (1) according to one of the above claims, characterized in that the communications set-up means (14) comprise a fastening means (19) designed to cooperate with the aperture (11) of the flexible inflatable band (10) in order to contribute, at least locally, to the attaching of the belt 2 to the flexible inflatable band 10.

7. Surgical ring (1) according to claim 6, characterized in that the fastening means (19) take the form of a button (19A) whose configuration is appreciably complementary to that of the aperture (11) of the flexible inflatable band (10), said aperture (11) forming a buttonhole designed to to receive said fastening means (19).

8. Surgical ring (1) according to one of the above claims, characterized in that the belt (2) has an internal face (2A) with a shape that is substantially complementary to that of the external face (10B) of the flexible inflatable band (10) against which said belt (2) is attached.

9. Surgical ring (1) according to one of the claims, characterized in that belt (2) is formed by a part made as a single piece comprising firstly on its internal face (2A) the hole (16) and secondly in the extension of one of its ends (4) the conduit (17) so as to form a single-piece belt (2).

10. Surgical ring (1) according to claims 2, 4 and 8, characterized in that the flexible inflatable band 10 has at least two ring-shaped gripping chambers 12, 13 which extend substantially in parallel to each other, the hole (16) of the belt (2) enabling communication of fluids between the flexible inflatable band (10) and the inflation fluid supply means 15

11. Surgical ring (1) according to claim 10, characterized in that the hole (16) of the belt (2) also provides for communication of fluids between each of the gripping chambers (12, 13).

12. Surgical ring (1) according to one of the above claims, characterized in that the flexible inflatable band (10) is formed by a part made as one piece so as to form a single-piece flexible inflatable band (10).

13. Surgical ring (1) according to one of the above claims, characterized in that it constitutes a gastric ring (1) designed to surround the stomach in order to reduce the section of passage of this stomach, in the context of a treatment for obesity.

14. Method for manufacturing a surgical ring (1) implantable in a human or animal body, said ring (1) being designed to go around a biological organ constituting a pocket or a duct in order to modify the section of passage of said organ, said method comprising a step for manufacturing a belt (2) comprising at least one means (3) for locking said ring (1) in a closed functional position about said biological ring, a step for manufacturing a flexible inflatable band (10) comprising at least one ring-shaped gripping chamber (12) designed to receive an inflation fluid and comprising at least one aperture (11) enabling the supply therein of inflation fluid, as well as a step for assembling the belt (2) with the flexible inflatable band (10), said belt (2) being brought to the flexible inflatable band (10) and fixed to it, said method for manufacturing being characterized in that the step for manufacturing the belt (2) comprises a sub-step for manufacturing a means (14) for placing said at least one aperture (11) of the flexible inflatable band (10) in communication with an inflation fluid supplying means (15).

15. Method for manufacturing according to claim 14, characterized in that the step for manufacturing the belt (2) comprises a sub-step for manufacturing the internal face (2A) of said belt (2), said internal face (2A) having a shape that is appreciably complementary to that of the external face (10B) of the flexible inflatable band (10) against which said internal face (2A) of the belt (2) is attached, said external face (10B) being manufactured during the step for manufacturing said inflatable band (10).

16. Method for manufacturing according to claim 14 or 15, characterized in that the step for manufacturing the belt (2) is performed by molding a part in one piece so as to form a single-piece belt (2) comprising firstly on its inner face (2A) a hole (16) enabling communication of fluids with said aperture (11) of the flexible inflatable band (10) and comprising secondly, in its extension, the communications set-up means (14) forming a conduit (17), said conduit (17) being connected firstly to the hole (16) and secondly to the inflation fluid supply means (15).

17. Method for manufacturing according to one of the claims 14 to 16, characterized in that the step for manufacturing the flexible inflatable band (10) is performed by molding a part in one piece so as to form a is single-piece inflatable band (10).

18. Method for manufacturing according to one of the claims 14 to 17, characterized in that it constitutes a method for manufacturing a gastric ring (1) designed to go around the stomach in order to reduce its section of passage in the context of a treatment for obesity.
